(19) United States
(12) Patent Application Publication
(10) Pub. No.: US 2016/0331574 A1
(43) Pub. Date: Nov. 17, 2016

(54) ORTHOPEDIC DEVICE AND METHOD FOR LOWER LIMB ELEVATION AND STABILIZATION

(71) Applicant: Bone Foam, Inc., Plymouth, MN (US)

(72) Inventors: Stephen Davis Lucey, Greensboro, NC (US); Chad L. Robran, Plymouth, MN (US)

(21) Appl. No.: 14/709,630

(22) Filed: May 12, 2015

(51) Int. Cl. A61F 5/37; A61G 7/075

(52) U.S. Cl. CPC A61F 5/37; A61G 7/0755

(57) ABSTRACT

In the context of medical recovery from injury and/or surgery to a lower extremity, a device may be used to provide elevation and support for an ankle along with restraint against knee rotation of a patient. The device may include a generally flat main body, two spaced-apart lateral ridges extending along or near lateral edges of the generally flat main body, and a medial depression between the lateral ridges that provides a support surface on which the ankle may be positioned.

### BACKGROUND

Following injury or a lower limb surgical procedure, a patient often lies or is placed in a supine position, i.e. with the front (anterior) portion of the lower limb facing up and the back portion of the lower limb (posterior) facing downward. As part of the recovery, it is often required that the lower limb remain in a certain position, being isolated, immobilized, and elevated, for a period of time.

Many kinds of undesirable movements are possible, however, during this time period of recovery. For example, natural movement may arise as the patient's foot naturally tends to rotate outwardly, which can exert torsion on an injured or newly repaired knee. Other movements may also be readily ascertained.

### BRIEF SUMMARY

In the context of medical recovery of extremity trauma, efforts may be taken to ensure that a patient's body is properly elevated, isolated, stabilized, and/or otherwise supported. The patient may lie, for example, in a supine position, with the ankle required to be isolated, elevated, immobilized, and supported. Furthermore, the natural inclination of the foot to rotate outward due to relaxation or gravity may in turn cause the tibia and fibula to rotate. This in turn causes unwanted and potentially dangerous torsion or torque to the knee joint. For example, a patient that just had knee surgery may incur serious pain, reinjure the knee, and even require more surgery. Thus, the foot may be required to be restricted from external rotation, or rotating laterally, away from the body.

A device may be used to provide elevation and support for an ankle along with restraint against foot and lower leg rotation. The patient may lie, for example, in a supine position, with the ankle resting on the device. The device may include a generally flat main body, two spaced-apart lateral ridges extending along or near lateral edges of the generally flat main body, and a medial depression between the lateral ridges that provides a support surface on which the ankle may be positioned. The device may further comprise a medial ridge connected to the lateral ridges such that a continuous ridge is formed.

### BRIEF DESCRIPTION OF THE DRAWINGS

FIG. 1 depicts a perspective view of a device for lower limb elevation and foot immobilization to prevent rotation.

FIG. 2 depicts a top view of a device for lower limb elevation and foot immobilization to prevent rotation.

FIG. 3 depicts a perspective view of a foot inserted into a device for lower limb elevation and foot immobilization to prevent rotation.

FIG. 4 depicts a perspective view of a device including a medial ridge for lower limb elevation and foot immobilization to prevent rotation.

FIG. 5 depicts a front view of a device for lower limb elevation and foot immobilization, the device including a medial ridge.

FIG. 6 depicts a perspective view of a foot inserted into a device for lower limb elevation and foot immobilization, the device including a medial ridge.

### DETAILED DESCRIPTION

For lower limb medical recovery, a patient often lies or is placed in a supine position, i.e. with the front (anterior) portion of the lower limb facing up and the back portion of the lower limb (posterior) facing down. As part of the recovery, it is often required that the lower limb remain in a certain position, being isolated and elevated, for a period of time. Also, elevating the lower leg from the bed surface may be desirable to ensure proper blood flow for reduction of edema and healing. Other benefits from elevating the leg may be realized. Rotation of the foot may cause rotation of the lower leg. The twisting or torsional effect on the leg may slow the process of healing, cause pain, and even further injury to the knee joint. Thus, support devices are needed to both elevate lower limbs and immobilize the foot to ensure proper positioning and proper healing.

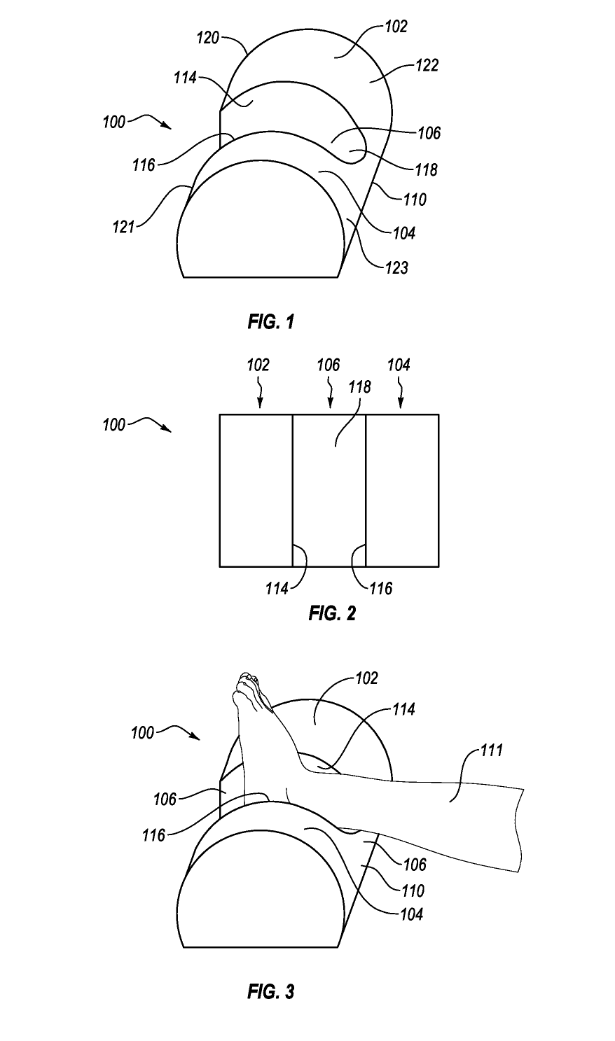

Turning to FIG. 1, a perspective view of a device 100 for lower limb elevation and stabilization is shown. The device 100 may comprise a generally flat main body 110, two spaced-apart lateral ridges 102 and 104 extending along or near lateral edges of the generally flat main body 100, and a medial depression 106 between the lateral ridges 102 and 104 that provides a support surface 118 on which the ankle may be positioned. The lateral ridges 102 and 104 may include inner sides 114 and 116 abutting support surface 118. The lateral ridges 102 and 104 further include proximal upper surfaces 120 and 121 and distal upper surfaces 122 and 123.

From the main body 110, the lateral ridges 102 and 104 extend vertically upward and are located near or at along sides of the main body 110. The lateral ridges 102 and 104 may span a desired length along the width of the main body 110, or span the entire side of the main body 110. Embodiments include that lateral ridges 102 and 104 have lengths that extend beyond one or more main body edges, creating an overhang wall formation.

As shown in FIG. 2, a top view of the device 100 is shown, with main body 110, lateral ridges 102 and 104, medial depression 106. The main body 110 may include a generally rectangular body. Alternatively, the body shape of the main body 110 may be multi-sided with sharp or rounded corners, such as rectangular, trapezoidal, or rectilinear, oblong, oval, circular, or have other shapes, proportions, and dimensions. The length may be larger than the width or, alternatively, the width may be larger than the length. Edges may be defined with sharp edges, curved edges, or a combination thereof.

The width, or wall thickness, of the lateral ridges 102 and 104 may be the same or they may vary. Although the width of the lateral ridges 102 and 104 are shown as being homogeneous in spanning the main body 110 in a parallel manner, the widths may have varying dimensions such that one or more of the inner sides 114 and 116 extends in a path that is not truly crosswise. For example, the widths of the inner sides 114 and 116 may be narrowed or tapered inward near or at the region where the ankle would be located on support surface 118. Alternatively, the widths of the inner sides 114 and 116 may be tapered outward from a center region where the ankle would be located on support surface 118.

The main body 110 may be contoured to conform to one or more of the lower leg, heel, ankle region, and talus of the patient. To conform to the lower leg, heel, ankle region, and talus of the patient, the inner sides 114 and 116 may have notches, or hollowed out cavities, removed near or at the center area where the ankle is located on the support surface 118 when placed in the device 100. Alternatively, a vertical hollowed out indention in each inner side 114 and 116 of the lateral ridges 102 and 104 may be provided, forming a vertical hollow on each inner side 114 and 116. Having a hollowed section, whether it be a cavity or vertical hollow, on each inner side 114 and 116 allows the device 100 to be used for either a left foot or right foot.

The support surface 118 of the medial depression 106 may be generally flat, however, embodiments include a variety of different surfaces, including a surface that is curved, rounded, wavy, concave, convex, slanted upward, curved upward, slanted downward, curved downward, as well as a variety of other surfaces. There may be a second depression within the depression 106, creating the effect that the heel is sunken into the second depression below the support surface level. This may help the heel to be further isolated from movement and further restrain leg and/or ankle rotation.

Embodiments include that at least a portion of a proximal upper surface of one of the spaced-apart lateral ridges 102 and 104 has a downward slope declining towards a proximal end. As shown, both proximal upper surfaces 120 and 121 have downward slopes, however, only one proximal upper surface may have a downward slope.

Embodiments include that at least a portion of a distal upper surface of one of the spaced-apart lateral ridges 102 and 104 has a downward slope declining towards a distal end. As shown, both distal upper surfaces 122 and 123 have downward slopes, however, only one distal upper surface may have a downward slope.

The downward slope may be a curved, or rounded, downward slope. The combined proximal and distal upper surfaces 122 and 123 may form an upside down cupping shape. Any suitable shape may be provided however. For example, instead of a curved shape, the combined upper surfaces may be flat, creating a standard wall appearance.

Outer edges of the proximal and distal upper surfaces 122 and 123 may be sharp, blunt, or be rounded and smoothed out. For height, the lateral ridges 102 and 104 may have the same or similar height, however, the heights may differ. Also, there may be differences in curvatures and edges, shapes, length, and width.

One or both of the lateral ridges 102 and 104 may extend generally vertically upward to a height of at least a general foot length according to anatomical measurements of a standard person. A standard person is a mathematical model of a person based on any suitable data that simulates a person's size, body proportions, and the like. The model can be based upon data, for example, used in the clothing and shoe industry to define sizes for apparel and the like. The standard person used and the data set used to derive the standard person is chosen with the user of the support device 100 in mind and can be based upon average values of body proportions from any sample of the population from, for example, total population, gender, age, body size or weight, nationality, or he like. The standard person may also be based upon any particular individual, or group of individuals. Thus, the standard person for a particular device 100 may be designed for marketing to the public in general, or be customized to fit a particular group of people, or to fit an individual.

Turning to FIG. 3, a foot 111 is shown inserted into the device 100 with the foot 111 resting on the support surface 118 of the medial depression 106 between lateral ridges 102 and 104. The foot 111 may face generally upward with toes pointing upward, as shown. However, the foot 111 may be tilted or angled with sides of the foot 111 resting against one of the inner sides 114 and 116. The lateral ridges 102 and 104 may be spaced to provide a tight fit or a relaxed fit. Furthermore, the medial depression 106 may be spaced with ample wiggle room to the extent that that the ankle need not even touch the inner sides 114 and 116.

Embodiments include that the support surface 118 and/or medial depression 106 have adjustments such that the space may be increased or decreased, as needed. For example, the lateral ridges 102 and 104 may be moved inward and outward along the main body 110 to form a smaller or larger space, respectively. Alternatively, inserts may be added and removed to either or both the inner sides 114 and 116 to change the space of the medial depression 106. Appropriate attachments for adjustments may include screws, clamps, straps, and other means commonly known in the art.

Also, materials may be added to provide further support, compression, structure, and weight. For example, cushioning may be added along the inner sides 114 and 116 of the lateral ridges 102 and 104 to provide a snug or tight fit when a foot 111 is inserted. Cushioning may be added in select areas along the inner sides 114 and 116 of the lateral ridges 102 and 104, for example, near or at the location where the ankle is anticipated to be placed. Alternatively, cushioning may be added on only one side, either the inner side 114 of the lateral ridge 102 or the inner side 116 of the lateral ridge 104. Again, cushioning may be added in select areas, either at the location or around the location where the ankle is anticipated to be placed or surrounding the location where the ankle is anticipated to be placed. Cushioning may be added with cutaways or surface definitions in the shape of a standard foot or ankle corresponding to various positions anticipated for the foot 111.

With the ankle in the device, it may be appreciated that rotational movements of the lower limb may be prevented. For example, the following movements may be stopped or prevented:
Dorsiflexion: Bending the foot at the ankle toward the shin (bending the foot upward).
Plantar flexion: Bending the foot at the ankle toward the sole (bending the foot downward).
Eversion: Turning the foot so the sole faces laterally.
Inversion: Turning the foot so the sole faces medially.
Circumduction: Moving a part so that its end follows a circular path (moving the toes in a circular motion without significantly moving the ankle).

In addition to preventing movements of the foot, the device may prevent internal and external rotational movements of the knee and the leg overall that would otherwise be caused by rotation of the foot. The leg may thus avoid torsional effects and torque caused by rotation of the foot. Also, the knee may be prevented from turning medially (inwardly) or laterally (outwardly) to face a direction other than a direction that is parallel to the direction of the foot.

In preventing rotation, a leg may be forced to stay in a generally fixed position such that ice may be applied to a region of the leg to reduce swelling or ease pain. In providing stabilizing support and isolation, the leg is more apt to get proper rest and healing. If the foot or leg should have to be moved, for example, to help adjust body position or remove the patient from the table, the device keeps the process simple because it is easy to put on and take off.

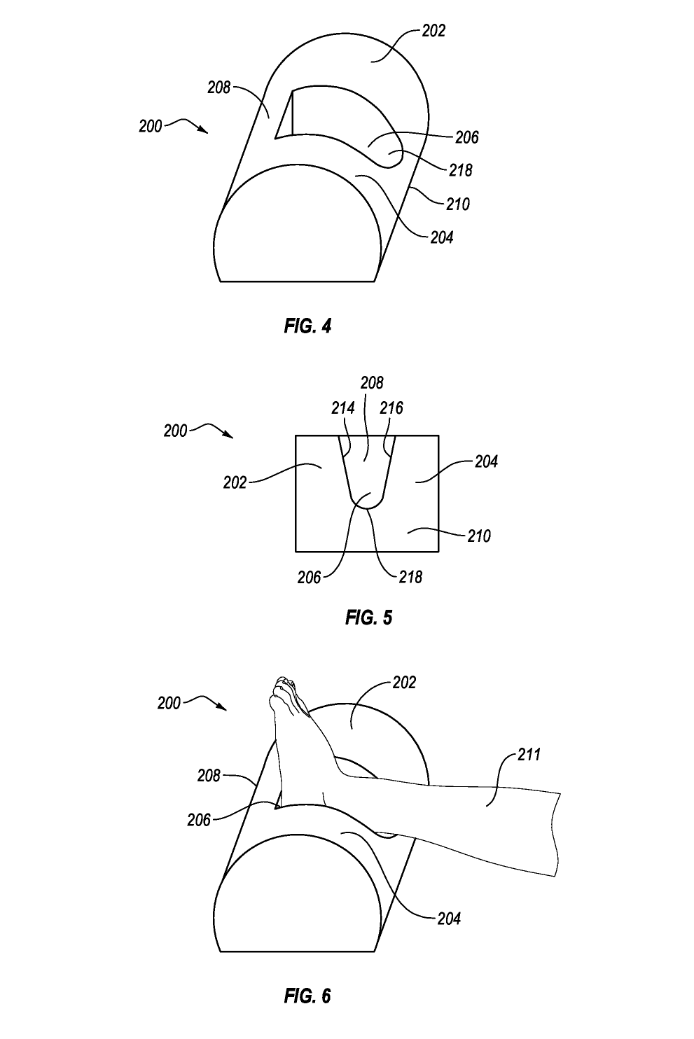

Turning to FIG. 4, a device 200 for lower limb elevation and stabilization is shown, including main body 210, lateral ridges 202 and 204, medial depression 206, medial ridge 208, and support surface 218. Embodiments include that the device 200 further comprise a medial ridge 208 extending along or near a distal end of the medial depression 206 to further support and restrain the patient's ankle. As shown, the medial ridge 208 at the medial section may be continuous with the lateral ridges 202 and 204 so as to form one medial ridge. Alternatives include, however, that the medial ridge 208 not be continuous with lateral ridges 202 and 204. The medial ridge 208 may have a similar width corresponding to widths of later ridges. Alternatively, the width of the medial ridge 208 may vary. The medial ridge 208 may have a similar height as shown, however, the heights may differ.

Turning to FIG. 5, device 200 is shown with lateral ridges 202 and 204 having angled inner sides 214 and 216 on opposing sides, as shown. The inner sides 214 and 216 taper downward and toward the center of the device. Alternatives include that the inner sides 214 and 216 may be rounded or vertical. With vertical inner sides and a flat surface, a box-like shape may be present.

As shown, the support surface 218 of the medial depression 206 may be curved, the curvature facing upward, and joining the angled inner sides 214 and 216. Alternatives include a flat support surface 218 that joins the angled inner sides 214 and 216.

The support surface 218, as well as other surfaces of the main body 210, may be smooth, pebbled, rough, textured, contoured, or have other features that improve and aid the user experience. For example, a textured surface may improve foot grip and thus further prevent movement.

Turning to FIG. 6, a foot 211 is shown inserted in the device 200. With the addition of medial ridge 208, the foot 211 is prevented from making movements such as plantar flexion movements. The addition of medial ridge 208 may help further prevent external and internal rotation of the knee joint. The medial ridge 208 may also aid the patient in inserting the foot 211 and removing the foot 211 from the device.

Many different materials may be used for the device. For example, the main body may comprise open cell polymer foam. Embodiments include that the polymer foam be coated with a flexible, fluid-impermeable polymer coating. Alternatives include that the device comprise radiolucent material.

With a given material, the main body may have a height and/or flexibility, coupled with sufficient firmness, so as to maintain a minimum elevation of the patient's ankle of one inch, from the upper surface of the supporting surface, during use. Providing stabilized elevation may be helpful in keeping the leg immobilized at a desired height during a period of medical recovery. Embodiments include that the main body have a firmness with sufficient yield to reduce pressure at and provide a comfortable support for high-pressure, soft-tissue areas.

Furthermore, the device may include an auxiliary pad configured to be positioned directly beneath the main body to further elevate the patient's ankle during use.

In using the device to elevate the patient's ankle during medical recovery, a stable platform, such as a bed or hospital bed, may be provided. The device may be placed on an upper surface of the stable platform. Proper positioning of the device may include putting the device underneath the ankle region of the patient, thereby elevating the ankle of the patient.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A device for supporting and elevating an ankle and restraining foot and leg rotation of a patient in a supine position, comprising:
a generally flat main body,
two spaced-apart lateral ridges extending along or near lateral edges of the generally flat main body,
a medial depression between the lateral ridges that provides a support surface on which the ankle may be positioned, and
wherein the lateral ridges confine and restrain a foot placed therein to limit or prevent rotation of the foot and leg.

2. A device as in claim 1, further comprising a ridge extending along or near a distal end of the medial depression to further restrain the patient's ankle.

3. A device as in claim 2, wherein the ridge at the medial section is continuous with the lateral ridges so as to form one continuous ridge.

4. A device as in claim 1, wherein the support surface is curved.

5. A device as in claim 1, wherein the support surface is generally flat.

6. A device as in claim 1, wherein at least a portion of a proximal upper surface of one of the spaced-apart lateral ridges has a downward slope declining towards a proximal end.

7. A device as in claim 1, wherein at least a portion of a distal upper surface of one of the spaced-apart lateral ridges has a downward slope declining towards a distal end.

8. A device as in claim 1, wherein the main body comprises open cell polymer foam.

9. A device as in claim 8, wherein the polymer foam is coated with a flexible, fluid-impermeable polymer coating.

10. A device as in claim 1, wherein the device comprises radiolucent material.

11. A device as in claim 1, wherein the main body has a height and/or flexibility, coupled with sufficient firmness, so as to maintain a minimum elevation of the patient's ankle of one inch, from an upper surface of a supporting surface, during use.

12. A device as in claim 10, wherein the main body has a firmness with sufficient yield to reduce pressure at and provide a comfortable support for high-pressure, soft-tissue areas.

13. A device as in claim 1, wherein the main body is contoured to approximately fit against one or more of the lower leg, heel, ankle region, and talus of the patient.

14. A device as in claim 1, further comprising an auxiliary pad configured to be positioned directly beneath the main body to further elevate the patient's ankle during use.

15. A device as in claim 1, further comprising a medial ridge which extends along or near a distal end of the medial depression to further support the foot and provide a surface which prevents plantar flexion of the foot.

16. A method for elevating and restraining a patient's ankle during a recuperative period, such as would be expected following a surgical procedure or injury, comprising:
providing a stable, relatively flat platform;
placing the device of claim 1 on an upper surface of said platform;
positioning the device underneath the ankle region of the patient, thereby elevating the ankle of the patient; and
limiting or preventing ankle and leg rotation to protect a knee of the patient.

17. A method as in claim 16, wherein the platform comprises a radiolucent surgical table.

18. A method as in claim 16, wherein the platform comprises a bed.
